(19) United States
(12) Patent Application Publication
(10) Pub. No.: US 2026/0175026 A1
(43) Pub. Date: Jun. 25, 2026

(54) DEVICES, SYSTEMS, AND METHODS FOR PROVIDING AFFERENT STIMULATION TO EVOKE RECTO-COLONIC REFLEX FOR COLONIC MOTILITY

(71) Applicants: The United States Government as represented by the Department of Veterans Affairs, Washington, DC (US); CASE WESTERN RESERVE UNIVERSITY, Cleveland, OH (US); THE METROHEALTH SYSTEM, Cleveland, OH (US)

(72) Inventors: Dennis Bourbeau, Washington, DC (US); Kenneth Gustafson, Washington, DC (US); Steven Brose, Washington, DC (US)

(21) Appl. No.: 19/125,641

(22) PCT Filed: Oct. 31, 2023
(86) PCT No.: PCT/US2023/036425

§ 371 Date: Apr. 29, 2025

Related U.S. Application Data
(60) Provisional application No. 63/421,024, filed on Oct. 31, 2022.

(51) Int. Cl. A61N 1/36; A61N 1/05

(52) U.S. Cl. CPC A61N 1/36007; A61N 1/0512; A61N 1/36034

(57) ABSTRACT

A system and method of use thereof for inducing colonic motility includes providing stimulations to a rectum of an individual. The stimulations include a plurality of low-frequency stimulations and a plurality high-frequency stimulations. The stimulations can be configured to activate sensory neurons and modulate the recto-colonic reflex pathway without directly activating motor neurons.

### CROSS-REFERENCE TO RELATED APPLICATION

This application claims priority to U.S. Provisional Application No. 63/421,024, filed Oct. 31, 2022, the entire disclosure of which is incorporated by reference herein.

### FIELD

The present disclosure is related to systems and methods of use thereof to treat individuals affiliated with slowed colonic mobility and, more specifically, to systems and methods for providing afferent electric stimulation to the rectum of an individual.

### BACKGROUND

The standard of care for individuals with slowed colonic motility, especially due to neurogenic bowel dysfunction, involves the design of a bowel program for predictable and effective elimination of the bowels. A bowel program includes diet, fluid intake, activity, and pharmaceutical or mechanical rectal stimulation. Approximately 80% of individuals with neurogenic bowel dysfunction use digital stimulation of the rectum to reflexively activate the distal colon, improve colonic motility, and facilitate bowel evacuation. However, the bowel routine can require over an hour to evacuate the bowels and this process often requires the assistance of a caregiver, which adds to the cost of care and reduces independence. Restoring bowel function is considered a high priority by individuals with paraplegia, but remains a critically unmet need requiring further development.

### BRIEF SUMMARY

Disclosed herein, in one aspect, is a method for inducing colonic motility including providing stimulations to a rectum of an individual. The stimulations include a plurality of low-frequency stimulations and a plurality of high-frequency stimulations.

In another aspect, a system includes at least one electrode and a signal generator in electrical communication with the at least one electrode. The signal generator is configured to cause the at least one electrode to provide a plurality of low-frequency stimulations and a plurality high-frequency stimulations.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 illustrates a known mechanical method for evoking the recto-colonic reflex.

FIG. 2 illustrates a system for providing electrical stimulation to evoke the recto-colonic reflex.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D. E, and F; and the example combination A-D. Likewise, any subset or combination of these is also specifically contemplated and disclosed. Thus, for example, the sub-group of A-E, B-F, and CE are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “an electrical pulse” can include a plurality of such electrical pulses, and reference to “the subject” is a reference to one or more subjects and equivalents thereof known to those skilled in the art, and so forth.

As used herein, the term “subject,” “patient,” or “individual” can be used interchangeably and refer to any organism to which an electrical pulse of this invention may be applied or administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like. The term does not denote a particular age or sex.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Systems and Methods for Providing Afferent Stimulation to Evoke Recto-Colonic Reflex

### Rectal Distension Evokes a Recto-Colonic Reflex to Load the Rectum

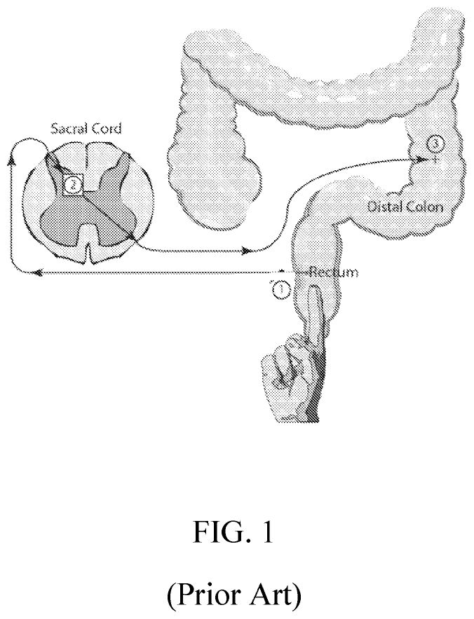

Referring to FIG. 1, digital rectal distension is the clinical standard for eliciting colon motility. The majority of individuals with neurogenic bowel dysfunction use digital rectal stimulation to mechanically distend the rectum and reflexively activate colon peristalsis, which loads stool into the rectum for bowel emptying. Digital rectal distension takes advantage of an existing reflex pathway affecting bowel function. The recto-colonic reflex has been well-studied in dogs, cats, and humans. Rectal distension activates mechanoreceptors innervating the rectum, which sends afferent information via small Aδ and C fibers to the sacral spinal cord. This input modulates a spinal reflex pathway that activates motor efferent drive to the colon, causing coordinated peristalsis and increasing colonic motility. As shown in FIG. 1, a gloved finger distends the rectum, which activates rectal afferents (1). Sensory information from the rectal afferents flows through the sacral posterior roots into the sacral spinal cord. Sacral spinal reflexes are modulated (2), activating colonic efferents that pass through the sacral anterior roots to evoke colonic motility (3).

Digital rectal stimulation is effective, but it requires an individual, or a caregiver, to reach into the individual's rectum with gloved fingers. Even with digital rectal stimulation and other bowel program tools, the bowel routine still typically requires an hour or two to complete. Reliance on digital rectal stimulation is associated with lost independence, lost time, lost dignity, and caregiver costs. As further disclosed herein, it is contemplated that substituting rectal distension for a method that does not include these drawbacks would provide positive and social and psychological benefits. As disclosed herein electrical stimulation can be used as an alternative to (or in addition to) mechanical stimulation to evoke the recto-colonic reflex and restore colonic motility.

### Colon Activity and Response to Mechanical and Electrical Stimulation

The slow action of the colon and its response to afferent mechanical and electrical stimulation in animals can be characterized. Three types of wave activity, including slow waves, fast waves, and tonic waves, can all occur spontaneously. Slow waves can be 0.6-7.5 cmH₂O in pressure and cycle 1-6 times per minute. Fast waves are larger and slightly faster than slow waves, with a pressure of 5-40 cmH₂O and frequency of 0.2-0.6 cycles per minute. These fast waves commonly occur and are associated with antiperistalsis (peristalsis in the oral direction). If fast waves and antiperistalsis is observed in response to stimulation, the stimulation parameters for possible application for fecal incontinence, which is another challenging clinical problem, can be determined. Tonic waves rarely occur spontaneously in the anaesthetized preparation. They can be 25-70 cmH₂O in amplitude and can last approximately 5-7 minutes in duration without necessarily repeating cyclically. These tonic waves include shortening and constriction of the colon and are associated with defecation. These are also the waves that are evoked in response to afferent stimulation, with stimulation typically applied for 1-2 minutes to evoke a tonic wave with an onset delay of approximately 1 minute. Peristaltic excretion can take several minutes.

### Afferent Electrical Stimulation Modulates Spinal Reflex Pathways to Restore Function

Neurons can be activated both mechanically and electrically. Sensors in the rectal wall respond to mechanical stretch, which activates the neurons innervating those sensors and initiates the recto-colonic reflex. Mechanical distension of the rectum can be substituted with electrical stimulation of rectal afferents. Electrical stimulation can activate the sensory (afferent) neurons without having to affect the sensors. Electrical stimulation may not activate the same neurons with the same pattern of neuronal behavior as mechanical distension, but the effect can be approximated by carefully choosing stimulus patterns and locations. The benefit to electrical stimulation for colon motility is that it can be fully implanted and can manage many of the drawbacks to digital rectal stimulation. For example, a fully implanted system would not require inserting an object into the anus and it would be controlled by an individual, rather than requiring a caregiver. Indeed, electrical stimulation has the potential to evoke the recto-colonic reflex more effectively and efficiently than digital rectal stimulation, which is subject to the skill of the caregiver and can require multiple attempts to elicit the reflex over a long period that increase the risk of hemorrhoids and rectal tissue damage.

An approach using electrical stimulation of sensory afferent nerves to restore pelvic function lost to neural trauma or disease is innovative. Investigations have recently made advances in afferent stimulation approaches for pelvic functions. Continuous sciatic and pudendal stimulation work has evaluated use of implanted electrodes on these nerves and on the S3 and S4 sacral nerve roots.

### Afferent Electrical Rectal Stimulation to Elicit Recto-Colonic Reflex

There are two approaches to electrically induce colonic motility: (a) motor (efferent) stimulation can directly activate the smooth muscles of the colon; and (b) electrical stimulation can also activate sensory afferents to affect motility. There are a number of potential benefits to using electrical rectal stimulation to activate rectal afferents to evoke the recto-colonic reflex and increase colonic motility. The greatest potential benefit is that electrical rectal stimulation can take advantage of a preserved functional reflex to produce coordinated colon peristalsis, as can be used clinically with digital rectal stimulation. The rectum is quite accessible and provides a relatively superficial site for electrode implantation, or for stimulation via inserted rectal probe. Indeed, patients can be screened before electrode implantation and this approach offers the ability to test electrical stimulation with a temporary, minimally invasive rectal probe. Efferent stimulation approaches have been tested and provide a viable backup approach if unforeseen problems occur with afferent stimulation.

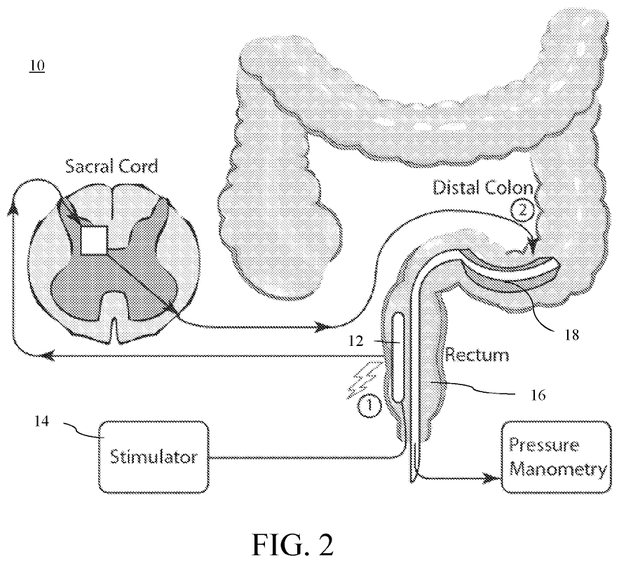

Referring to FIG. 2, an implanted system can replace rectal distension to improve colonic motility. The implanted system can be operated by a user (e.g., by operation of a push-button in communication with the implanted system).

### Electrical Stimulation of the Colon Increases Colonic Motility

Currently, there are no clinically available interventions on the US market for neurogenic bowel dysfunction that use electrical stimulation. However, bowel function can be improved with electrical stimulation. Stimulation of the colon have been performed to directly activate the smooth muscle of the colon and move fecal contents in animals. Previous attempts at electrical stimulation were prone to a number of potential drawbacks. Colon stimulation typically requires multiple electrodes along the colon to activate segments of the colon to approximate coordinated peristalsis. In some exemplary embodiments, an electrode was placed in the distal colon, which is innervated by the same nerves as the rectum. Thus, it is contemplated that colon motility can be evoked by this single distal colon electrode modulating an excitatory reflex pathway. Colon stimulation as an approach has demonstrated improved colon motility. However, because the mechanism underlying colon stimulation is not well understood and is not anchored by the clinical standard of the mechanically-induced recto-colonic reflex, electrical rectal stimulation can be preferable to electrical colon stimulation.

### Sacral Root Stimulation

Two clinically tested approaches that activate the smooth muscle of the bowel include electrodes implanted (1) in the sacral foramen for sacral neuromodulation or (2) on the sacral anterior roots, which activates motor efferents and directly drives colon activity. The Interstim System (Medtronic, Minneapolis, MN) has been shown to improve fecal incontinence for individuals without spinal cord injuries. Some of the clinical testing of this system has been conducted. However, this approach has not demonstrated effectiveness for improving neurogenic bowel dysfunction. Sacral anterior root stimulation is an implanted system that can activate the colon and has shown decreased defecation times, and human testing of the Brindley system suggests that stimulation improves bowel evacuation in individuals with spinal cord injury. Sacral anterior root stimulation triggers unwanted external anal sphincter contractions that can prevent efficient bowel emptying, as well as external urethral sphincter reflexes that affect the efficiency of bladder emptying. Typically, a posterior rhizotomy is performed wherein the sacral posterior roots carrying sensory information to the spinal cord are transected, thus removing these reflexes. Though this posterior rhizotomy is effective, individuals do not prefer it because it can abolish remaining pelvic sensation and desirable reflexes, including reflex sexual functions such as erection.

In some aspects, the unwanted activity can be inhibited without a dorsal rhizotomy. Afferent stimulation and kilohertz high frequency nerve block have been performed in chronic spinalized animals to inhibit the external urethral sphincter. The same or similar stimulation can be used with sacral root stimulation for colon activation.

### Exemplary Stimulation Systems and Methods

FIG. 2 illustrates electrical stimulation of the sensory nerves innervating the lower colon and rectum. As shown, the electrical stimulation system 10 may include at least one electrode 12 positioned within the rectum 16 of the patient, and a signal generator 14 in electrical communication with the at least one electrode 12. The electrical stimulation system 10 may be used to impart a pattern of stimulation that can include a slow carrier frequency. The slow carrier frequency can be from about % Hz to about 5 Hz. In some optional aspects, the slow carrier frequency can be from about 0.5 Hz to about 2 Hz. The pattern of stimulation can further include bursts of pulses (e.g., from about 5 pulses to about 100 pulses, or from about 10 pulses to about 50 pulses). Within each burst (e.g., sequence), pulses can be provided at a frequency from about 25 Hz to about 500 Hz. In some optional aspects, within each burst, pulses can be provided at a frequency from about 50 Hz to about 200 Hz. This pattern can be sufficient to activate sensory neurons and modulate the recto-colonic reflex pathway without directly activating the motor neurons.

Optionally, as shown in FIG. 2, the electrical stimulation system 10 can comprise a pressure manometry apparatus including a balloon catheter 18 that is inserted into the colon, just past the sigmoid colon. In use, it is contemplated that the pressure manometry apparatus can measure and/or provide an indication of colonic pressure and/or motility. During typical usage of the electrical stimulation system 10, it is contemplated that the pressure manometry apparatus can be omitted. However, it is contemplated that the pressure manometry apparatus can be provided when a clinician wishes to evaluate the effectiveness of the electrical stimulation system 10 at modulating the recto-colonic reflex pathway.

An exemplary method comprises providing stimulations to a rectum of an individual. The stimulations can comprise a plurality of low-frequency stimulations and a plurality high-frequency stimulations. The stimulations can be configured to activate sensory neurons and modulate the recto-colonic reflex pathway (schematically depicted as a lightning bolt in FIG. 2) without directly activating the motor neurons. Specifically, it is contemplated that electrical stimulation of the rectum as disclosed herein can modulate sacral spinal reflexes, thereby activating colonic efferents that pass through the sacral anterior roots to evoke and/or restore colonic motility (3).

In some aspects, the low frequency stimulations have a frequency from about ¼ Hz to about 5 Hz, or from about 0.5 Hz to about 2 Hz.

In some aspects, the high frequency stimulations have a frequency from about 25 Hz to about 500 Hz, or from about 50 Hz to about 200 Hz.

In some aspects, the plurality of high-frequency stimulations comprise, or consist of, about 5 sequential pulses to about 100 sequential pulses. In further aspects, the plurality of high-frequency stimulations comprise, or consist of, about 10 sequential pulses to about 50 sequential pulses

In some aspects, the plurality of high frequency stimulations can be provided as at least one sequence of high frequency stimulations interlaced with the plurality of low-frequency stimulations.

In other aspects, the plurality of high frequency stimulations can be provided as at least one sequence of high frequency stimulations delivered in between consecutive sequences of low-frequency stimulations.

The disclosed systems and methods may be utilized to improve the colonic motility of an individual by way of electrical stimulation of the rectum. For example, an individual suffering over a two year period from a spinal cord injury followed a bowel emptying routine that consisted of digital rectal stimulation, as is known in the art. Specifically, the individual's routine would frequently require three to four bouts of digital rectal stimulation at 30 minute intervals, meaning the full emptying of the bowel would frequently occur over a two hour period. Additionally, caregiver assistance was required. In contrast, referring again to FIG. 2, electrical rectal stimulation, such as with an electrode 12 of the present electrical stimulation system 10, was found to allow for emptying of the bowel after only one bout of electrical stimulation of the rectum in over half of the trials. Although some of the trials were found to require two bouts of electrical stimulation for emptying of the bowel, electrical rectal stimulation was found to decrease the overall time required for the emptying of the bowel and required little to no caregiver assistance.

In some aspects, at least one electrode 12 can be implanted into the rectum of the individual for providing the stimulations to the rectum.

As noted, the disclosed electrical stimulation system 10 can comprise at least one electrode 12 and a signal generator 14 in electrical communication with the at least one electrode 12. The signal generator can be configured to cause the at least one electrode to provide: a plurality of low-frequency stimulations and a plurality high-frequency stimulations.

In some aspects, the at least one electrode comprises a large surface area electrode. For example, in some aspects, the large surface area electrode has a surface area of at least 1 square centimeter. In further aspects, the large surface area electrode can have a surface area of at least 1.5 square centimeters, from 0.8 to 2 square centimeters, at least 2 square centimeters, or at least 3 square centimeters.

The system can further comprise one or a plurality of actuators in communication with the signal generator. In some aspects, the actuator(s) can comprise one or a plurality of push-buttons, a touchscreen, or other structure that is configured to receive a user input. Optionally, the actuator(s) can be provided in association with a user input device, such as a touchscreen, a tablet, a remote control, or other device that is communicatively coupled (e.g., electrically coupled) to the signal generator.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A system comprising:
at least one electrode configured for positioning within a rectum of an individual;
a signal generator in electrical communication with the at least one electrode, wherein the signal generator is configured to cause the at least one electrode to provide:
   a plurality of low-frequency stimulations to the rectum; and
   a plurality of high-frequency stimulations to the rectum,
wherein the plurality of low-frequency stimulations and the plurality of high-frequency stimulations are configured to activate sensory neurons of the individual and modulate the recto-colonic reflex pathway of the individual.

Aspect 2: The system of aspect 1, wherein the low frequency stimulations have a frequency from about 14 Hz to about 5 Hz.

Aspect 3: The system of aspect 2, wherein the low frequency stimulations have a frequency from about 0.5 Hz to about 2 Hz.

Aspect 4: The system of any one of aspects 1-3, wherein the high frequency stimulations have a frequency from about 25 Hz to about 500 Hz.

Aspect 5: The system of aspect 4, wherein the high frequency stimulations have a frequency from about 50 Hz to about 200 Hz.

Aspect 6: The system of any one of aspects 1-5, wherein the plurality of high-frequency stimulations comprise about 5 sequential pulses to about 100 sequential pulses.

Aspect 7: The system of aspect 6, wherein a single series of the plurality of high-frequency stimulations consists of about 5 sequential pulses to about 100 sequential pulses.

Aspect 8: The system of aspect 6 or aspect 7, wherein the plurality of high-frequency stimulations comprise about 10 sequential pulses to about 50 sequential pulses.

Aspect 9: The system of aspect 8, wherein a single series of the plurality of high-frequency stimulations consists of about 10 sequential pulses to about 50 sequential pulses.

Aspect 10: The system of any one of aspects 1-9, wherein the plurality of high frequency stimulations are provided as at least one sequence of high frequency stimulations interlaced with the plurality of low-frequency stimulations.

Aspect 11: The system of any one of aspects 1-10, wherein the plurality of high frequency stimulations are provided as at least one sequence of high frequency stimulations delivered in between consecutive sequences of low-frequency stimulations.

Aspect 12: The system of any one of aspects 1-11, wherein the at least one electrode comprises a large surface area electrode.

Aspect 13: The system of aspect 12, wherein the large surface area electrode has a surface area of at least 1 square centimeter.

Aspect 14: The system of any one of aspects 1-13, further comprising an actuator in communication with the signal generator.

Aspect 15: The system of aspect 14, wherein the actuator comprises a push-button.

Aspect 16: A method comprising:
providing stimulations to a rectum of an individual, the stimulations comprising:
a plurality of low-frequency stimulations; and
a plurality of high-frequency stimulations.

Aspect 17: The method of aspect 16, wherein the low frequency stimulations have a frequency from about 14 Hz to about 5 Hz.

Aspect 18: The method of aspect 17, wherein the low frequency stimulations have a frequency from about 0.5 Hz to about 2 Hz.

Aspect 19: The method of any one of the preceding aspects, wherein the high frequency stimulations have a frequency from about 25 Hz to about 500 Hz.

Aspect 20: The method of aspect 19, wherein the high frequency stimulations have a frequency from about 50 Hz to about 200 Hz.

Aspect 21: The method of any one of the preceding aspects, wherein the plurality of high-frequency stimulations comprise about 5 sequential pulses to about 100 sequential pulses.

Aspect 22: The method of aspect 21, wherein a single series of the plurality of high-frequency stimulations consists of about 5 sequential pulses to about 100 sequential pulses.

Aspect 23: The method of aspect 21 or aspect 22, wherein the plurality of high-frequency stimulations comprise about 10 sequential pulses to about 50 sequential pulses.

Aspect 24: The method of aspect 23, wherein a single series of the plurality of high-frequency stimulations consists of about 10 sequential pulses to about 50 sequential pulses.

Aspect 25: The method of any one of the preceding aspects, wherein the plurality of high frequency stimulations are provided as at least one sequence of high frequency stimulations interlaced with the plurality of low-frequency stimulations.

Aspect 26: The method of any one of the preceding aspects, wherein the plurality of high frequency stimulations are provided as at least one sequence of high frequency stimulations delivered in between consecutive sequences of low-frequency stimulations.

Aspect 27: The method of any one of the preceding aspects, further comprising: implanting at least one electrode into the rectum of the individual, wherein providing stimulations to the rectum of the individual comprises using the at least one electrode to provide the stimulations.

Aspect 28: The method of aspect 27, wherein the stimulations activate sensory neurons and modulate a recto-colonic reflex pathway of the individual without directly activating motor neurons of the individual.

Those skilled in the art will recognize, or be able to ascertain using no more than routine experimentation, many equivalents to the specific embodiments of the method and compositions described herein. Such equivalents are intended to be encompassed by the following claims.

### REFERENCES

The following references are hereby incorporated by reference herein in their respective entireties:
1. King R B, Parsons K C, et al. Neurogenic Bowel Management in Adults with Spinal Cord Injury.; 1998.
2. Anderson K D, Borisoff J F. Johnson R D. Stiens S a. Elliott S L. Long-term effects of spinal cord injury on sexual function in men: implications for neuroplasticity. Spinal Cord. 2007; 45:338-348.
3. Anderson K D. Targeting recovery: priorities of the spinal cord-injured population. J Neurotrauma. 2004; 21(10):1371-1383.
4. Fukuda H, Fukai K. The afferent and efferent pathways of the recto-colonic reflex in the dog. Jpn J Physiol. 1985; 35(5):795-801.
5. Chen J H, Sallam H S, Lin L, Chen J D Z. Colorectal and rectocolonic reflexes in canines: involvement of tone, compliance, and anal sphincter relaxation. Am J Physiol Regul Integr Comp Physiol. 2010; 299(3):R953-9.
6. Shafik A. Shafik A A, El-Sibai O, Ahmed I. Role of sympathetic innervation in the defecation mechanism: a novel concept of its function. J Spinal Cord Med. 2003; 26(2):150-154.
7. Garrett B J R, Howard E R, Jones W. The internal anal sphincter in the cat: A study of nervous mechanisms affecting tone and reflex activity. J Physiol. 1974; 243:153-166.
8. Bouvier M, Gonella J. Nervous control of the internal anal sphincter of the cat. J Physiol. 1981; 310:457-469.
9. Ruhl A, Thewiûen M, Ross H G, Cleveland S, Frieling T, Enck P. Discharge patterns of intramural mechanoreceptive afferents during selective distension of the cat's rectum. Neurogastroenterol Motil. 1998:10:219-225.
10. Sjöqvist A, Hellström P M, Jodal M, Lundgren O. Neurotransmitters involved in the colonic contraction and vasodilatation elicited by activation of the pelvic nerves in the cat. Gastroenterology. 1984; 86(6):1481-1487.
11. Takaki M, Neya T. Nakayama S. Functional role of lumbar sympathetic nerves and supraspinal mechanism in the defecation reflex of the cat. Acta Med Okayama. 1987; 41(6):249-257.
12. Buntzen S, Nordgren S, Delbro D, Hulten L. Reflex interaction from the urinary bladder and the rectum on anal motility in the cat. J Auton Nerv Svst. 1995; 54:33-40.
13. de Groat W, Krier J. An electrophysiological study of the sacral parasympathetic pathway to the colon of the cat. J Physiol. 1976:260:425-445.
14. de Groat W, Krier J. The sacral parasympathetic reflex pathway regulating colonic motility and defaecation in the cat. J Physiol. 1978; 276:481-500.
15. Frost F. Hartwig D, Jaeger R, Leffler E, Wu Y. Electrical stimulation of the sacral dermatomes in spinal cord injury: effect on rectal manometry and bowel emptying. Arch Phys Med Rehabil. 1993; 74:696-701.
16. Shafik A. Recto-colic reflex: role in the defecation mechanism. Int Surg. 81(3):292-294.
17. Ng C, Danta M, Prott G, Badcock C A, Kellow J, Malcolm A. Modulatory influences on antegrade and retrograde tonic reflexes in the colon and rectum. Am J Physiol Gastrointest Liver Physiol. 2004; 287(5):G962-6.
18. Possover M, Schurch B, Henle K P. New strategies of pelvic nerves stimulation for recovery of pelvic visceral functions and locomotion in paraplegics. Neurourol Urodvn. 2010; 29:1433-1438.
19. Worsoe J, Fynne L, Laurberg S, Krogh K, Rijkhoff N J M. Electrical stimulation of the dorsal clitoral nerve reduces incontinence episodes in idiopathic faecal incontinent patients: a pilot study. Colorectal Dis. 2012; 14(3):349-355.
20. Worsoe J, Fynne L, Laurberg S, Krogh K, Rijkhoff N J M. Acute effect of electrical stimulation of the dorsal genital nerve on rectal capacity in patients with spinal cord injury. Spinal Cord. 2012; 50(6):462-466.
21. Bruninga K, Riedy L, Keshavarzian A, Walter J. The effect of electrical stimulation on colonic transit following spinal cord injury in cats. Spinal Cord. 1998:36:847-853.
22. Tai C, Booth a M, de Groat W C, Roppolo J R. Colon and anal sphincter contractions evoked by microstimulation of the sacral spinal cord in cats. Brain Res. 2001; 889(1-2):38-48.
23. Sevcencu C, Rijkhoff N J M, Sinkjaer T. Colon emptying induced by sequential electrical stimulation in rats. IEEE Trans Neural Syst Rehabil Eng Publ IEEE Eng Med Biol Soc. 2005; 13(4):516-523.
24. El-Gazzaz G, Zutshi M, Salcedo L, Hammel J, Rackley R, Hull T. Sacral neuromodulation for the treatment of fecal incontinence and urinary incontinence in female patients: long-term follow-up. Int J Colorectal Dis. 2009; 24:1377-1381.
25. Wexner S D, Coller J a, Devroede G, et al. Sacral nerve stimulation for fecal incontinence: results of a 120-patient prospective multicenter study. Ann Surg. 2010; 251(3):441-449.
26. Brindley G S. An implant to empty the bladder or close the urethra. J Neurol Neurosurg Psychiatry. 1977:40(4):358-369.
27. Binnie N R. Smith A N. Creasey G H. Edmond P. Constipation associated with chronic spinal cord injury: The effect of pelvic parasympathetic stimulation by the Brindley stimulator. Paraplegia. 1991; 29:463-469.
28. Varma J S, Binnie N, Smith A N, Creasey G H, Edmond P. Differential effects of sacral anterior root stimulation on anal sphincter and colorectal motility in spinally injured man. Br J Surg. 1986; 73(6):478-482.
29. Creasey G H, Grill J H, Korsten M, et al. An implantable neuroprosthesis for restoring bladder and bowel control to patients with spinal cord injuries: a multicenter trial. Arch Phys Med Rehabil. 2001; 82(11):1512-1519.
30. Sanders P M H, Ijzerman M J, Roach M J, Gustafson K J. Patient preferences for next generation neural prostheses to restore bladder function. Spinal Cord. 2011; 49(1):113-119.
31. McCoin J, Bhadra N, Gustafson K J. Electrical stimulation of sacral dermatomes can suppress aberrant urethral reflexes in felines with chronic spinal cord injury. Neurourol Urodyn. 2013; 32(1):92-97.
32. Franke M, Vrabec T, Wainright J, Bhadra N, Bhadra N, Kilgore K. Combined KHFAC+DC nerve block without onset or reduced nerve conductivity after block. J Neural Eng. 2014; 11(5):056012.

1. A system comprising:
at least one electrode configured for positioning within a rectum of an individual;
a signal generator in electrical communication with the at least one electrode, wherein the signal generator is configured to cause the at least one electrode to provide:
a plurality of low-frequency stimulations to the rectum; and
a plurality of high-frequency stimulations to the rectum,
wherein the plurality of low-frequency stimulations and the plurality of high-frequency stimulations are configured to activate sensory neurons of the individual and modulate the recto-colonic reflex pathway of the individual.

2. The system of claim 1, wherein the low frequency stimulations have a frequency from about ¼ Hz to about 5 Hz.

3. The system of claim 2, wherein the low frequency stimulations have a frequency from about 0.5 Hz to about 2 Hz.

4. The system of claim 1, wherein the high frequency stimulations have a frequency from about 25 Hz to about 500 Hz.

5. The system of claim 4, wherein the high frequency stimulations have a frequency from about 50 Hz to about 200 Hz.

6. The system of claim 1, wherein the plurality of high-frequency stimulations comprise about 5 sequential pulses to about 100 sequential pulses.

7. (canceled)

8. The system of claim 6 wherein the plurality of high-frequency stimulations comprise about 10 sequential pulses to about 50 sequential pulses.

9. (canceled)

10. The system of claim 1, wherein the signal generator is configured to provide the plurality of high frequency stimulations as at least one sequence of high frequency stimulations interlaced with the plurality of low-frequency stimulations.

11. The system of claim 1, wherein the signal generator is configured to provide the plurality of high frequency stimulations as at least one sequence of high frequency stimulations delivered in between consecutive sequences of low-frequency stimulations.

12. The system of claim 1, wherein the at least one electrode comprises a large surface area electrode having a surface area of at least one square centimeter.

13. (canceled)

14. The system of claim 1, further comprising an actuator in communication with the signal generator.

15. (canceled)

16. A method comprising:
positioning the at least one electrode of the system of claim 1 in a rectum of an individual;
causing, by the signal generator of the system, the at least one electrode to provide electrical stimulations to the rectum of the individual, the stimulations comprising:
a plurality of low-frequency stimulations; and
a plurality of high-frequency stimulations.

17. The method of claim 16, wherein the low frequency stimulations have a frequency from about ¼ Hz to about 5 Hz.

18. The method of claim 17, wherein the low frequency stimulations have a frequency from about 0.5 Hz to about 2 Hz.

19. The method of claim 16, wherein the high frequency stimulations have a frequency from about 25 Hz to about 500 Hz.

20. The method of claim 19, wherein the high frequency stimulations have a frequency from about 50 Hz to about 200 Hz.

21. The method of claim 16, wherein the plurality of high-frequency stimulations comprise about 5 sequential pulses to about 100 sequential pulses.

22. (canceled)

23. The method of claim 21, wherein the plurality of high-frequency stimulations comprise about 10 sequential pulses to about 50 sequential pulses.

24. (canceled)

25. The method of claim 16, wherein the plurality of high frequency stimulations are provided as at least one sequence of high frequency stimulations interlaced with the plurality of low-frequency stimulations.

26. The method of claim 16, wherein the plurality of high frequency stimulations are provided as at least one sequence of high frequency stimulations delivered in between consecutive sequences of low-frequency stimulations.

27. The method of claim 16, wherein the stimulations activate sensory neurons and modulate a recto-colonic reflex pathway of the individual.

28. The method of claim 27, wherein the stimulations do not directly activate motor neurons of the individual.
